(19) United States
(12) Patent Application Publication
(10) Pub. No.: US 2012/0264202 A1
(43) Pub. Date: Oct. 18, 2012

(54) SYSTEM FOR PERFORMING POLYMERASE CHAIN REACTION NUCLEIC ACID AMPLIFICATION

(76) Inventors: Christopher I. WALKER, PALO ALTO, CA (US); Aditya RAJAGOPAL, IRVINE, CA (US)

(21) Appl. No.: 13/423,674

(22) Filed: Mar. 19, 2012

Related U.S. Application Data
(60) Provisional application No. 61/466,835, filed on Mar. 23, 2011.

(51) Int. Cl. C12M 1/40; B05D 1/36; B05D 3/00; B05D 5/12

(52) U.S. Cl. 435/287.2; 435/289.1; 427/58

(57) ABSTRACT

A printed circuit structure containing a fluidic chamber configured to receive an aqueous solution containing a sample to be analyzed and fluorophore for polymerase chain reaction analysis. The printed circuit structure also contains a heating element that provides for temperature cycling of the fluidic chamber to support polymerase chain reaction analysis.

### CROSS-REFERENCE TO RELATED APPLICATIONS

The present application is related to and claims the benefit of the following copending and commonly assigned U.S. patent applications: U.S. Patent Application No. 61/466,835, titled “Monolithic Miniaturized System for Performing Polymerase Chain Reaction Nucleic Acid Amplification Fabricated within a Printed Circuit Board,” filed on Mar. 23, 2011; the entire contents of this application are incorporated herein by reference.

### BACKGROUND

1. Field

The present disclosure relates to real-time polymerase chain reaction analysis. More specifically, the present disclosure describes a printed circuit structure providing a fluidic structure configured to receive an aqueous solution containing a sample to be analyzed and fluorophore. The printed circuit structure provides for temperature cycling of the fluidic chamber to support polymerase chain reaction analysis.

2. Description of Related Art

DNA/RNA analysis is an increasingly important analysis tool for a wide variety of biochemical applications. The uniqueness of nucleic acid sequences allows for the detection of biological agents with a high degree of specificity. However, since the concentration of DNA in biologically derived analytes is low, the DNA concentration must be increased, i.e., amplified, to be effectively used as a detection tool. The dominant method to amplify DNA concentration is the polymerase chain reaction (PCR). In PCR, a DNA-containing solution is mixed with primer strands that bracket the desired sequence, free nucleotides (the building blocks of a DNA strand), a polymerase enzyme, and buffer solution. The resulting solution is then cycled through a series of temperatures, which allow the DNA to separate (“melt”), and polymerize a replicated strand from the available free nucleotides. Depending on the type of polymerase employed, the temperature steps are typically 95° C. (unwinding the DNA, or melting), 50°-65° C. (attracting free nucleotides, or annealing), and 70° C. (replicating the DNA strand—polymerization). With each temperature cycle, the total concentration of the desired DNA sequence doubles, allowing the concentration to be exponentially amplified by a series of temperature cycles.

When a fluorophore (fluorescent chemical) linked to a specific target DNA sequence is added to the solution, and the solution illuminated during the temperature cycling process, this fluorescence of the solution becomes proportional to the total concentration of the targeted DNA sequence. This is known as real time PCR (RT-PCR). RT PCR allows for the optical detection of biological agents (a strain of E. Coli, for example) with near perfect specificity from samples in which the initial concentration of the agent is very low (a few cells). It is thus ideal for a variety of applications such as disease detection.

Most commercially available instrumentation for PCR/RT-PCR relies on bulk samples of liquid. Solutions are typically manipulated in small vials/cuvettes/capillary tubes with volumes ranging from the hundreds of microliters to milliliters. Systems which perform PCR on very small volumes (a microliter) are commercially available (Fluidigm), but the instrumentation is typically optimized to process large numbers of discrete samples in parallel. PCR instrumentation thus tends to be used in a stationary lab environment.

Real time DNA/RNA analysis in a field environment would be advantageous for the identification of biological agents, since such analysis would avoid the delays inherent in returning samples to a laboratory. Analysis systems that could be provided at a low cost would allow for the wide use of such systems, which also allow for the avoidance of delays inherent in returning samples to a laboratory. Therefore, there exists a need in the art for performing RT-PCR in a field environment at a relatively low cost.

### SUMMARY

Described herein are devices, apparatus, methods, arrays, and systems according to embodiments of the present invention that provide for performing RT-PCR in a field environment. Miniaturization of the functional components (liquid container, heater, cooler; optics) used for RT-PCR supports field environment testing, especially of those components can be provided at low cost. Fabrication of the central components—a fluid container and heater—with the techniques developed for printed circuit boards allows for a low cost RT-PCR/PCR cartridge.

A first exemplary embodiment is a printed circuit board structure comprising: a first layer; a second layer disposed on the first layer, wherein the second layer comprises one or more electrical interconnections; and a third layer disposed on the first layer or the second layer or on the first and second layer, wherein the third layer comprises an enclosed planar chamber, wherein the enclosed planer chamber is configured to receive an aqueous solution. The enclosed planar chamber may be formed by depositing metal in the third layer and then removing the metal by an etch removal process. The third layer may comprise optically transparent material, where the third layer material may be polyimide. The second layer may comprise a heating element, where the heating element may comprise one or more traces having a serpentine path from an electrical source to an electrical return. A heat spreading element may be disposed between the heating element and the enclosed planer chamber and the heat spreading element may comprise a metal layer. A lyophilized polymerase chain reaction solution may be contained within the enclosed planar structure.

A second exemplary embodiment is a system for real time polymerase chain reaction analysis comprising: a printed circuit board cartridge comprising: a first layer comprising a heating element; and a second layer in thermal communication with the first layer, wherein the second layer comprises an enclosed planar chamber having an optically accessible outer face, wherein the enclosed planer chamber is configured to receive an aqueous solution, an optical source configured to direct optical energy into the enclosed planer chamber; and, an optical monitor configured to monitor optical energy radiated from the enclosed planer chamber. An electrical source may be coupled to the heating element, where the electrical source is controlled to control temperature of the enclosed planer chamber. The electrical source may be controlled to cycle temperature of the enclosed planer chamber through selected temperatures. A heat spreading element may be disposed between the heating element and the enclosed planer chamber. The heating element may comprise one or more traces having a serpentine path from an electrical source to an electrical return. The heat spreading element may comprise a metal layer. The printed circuit board structure may have a lyophilized polymerase chain reaction solution contained within the enclosed planar structure.

A third exemplary embodiment is a method for forming a temperature controlled fluidic chamber comprising: depositing an electrical layer on a base layer to form a resistive heating element; depositing a polyimide layer on the base layer or the electrical layer or the base layer and the electrical layer; depositing metal within the polyimide layer to form a planar structure; and removing the metal from the planar structure to form a planar chamber within the polyimide layer. The resistive heating element may comprise one or more serpentine metal traces The method may further comprise depositing a metal layer between the resistive heating element and the planer structure. The method may comprise forming a temperature controlled fluidic chamber for polymerase chain reaction analysis, where the method further comprises directing a polymerase chain reaction solution into the planar chamber and lyophilizing the polymerase chain reaction solution.

### BRIEF DESCRIPTION OF THE SEVERAL VIEWS OF THE DRAWINGS

FIG. 1A illustrates an initial printed circuit board structure which supports creation of a fluidic chamber.

FIG. 1B illustrates the deposition of a mask layer on the structure depicted in FIG. 1A.

FIG. 1C shows the formation of a fluidic chamber.

FIG. 1D depicts the final configuration of a printed circuit board structure with a fluidic chamber.

FIG. 2 illustrates a circuit board trace for creating a heating element.

FIG. 3 shows a heating structure disposed beneath a fluidic chamber in a printed circuit board structure.

FIG. 4 shows a heating spreading element beneath a heating element and a fluidic chamber in a printed circuit board structure.

FIG. 5 shows a real-time polymerase chain reaction analysis system.

FIG. 6 shows a printed circuit board structure with a heating element and a fluidic chamber.

FIG. 7 shows a printed circuit board structure with a heating element and a heat spreading element.

FIG. 8 shows temperature curves for heating provided by an exemplary printed circuit board structure.

FIG. 9 shows temperature curves for cooling provided by an exemplary printed circuit board structure.

### DETAILED DESCRIPTION

The present disclosure describes the provision of RT-PCR capability through the utilization of printed circuit board technology. Miniaturization of the functional components (liquid container, heater, cooler; optics) used for RT-PCR supports field environment testing, especially if those components can be provided at low cost. Fabrication of the central components—a fluid container and heater—with the techniques developed for printed circuit boards allows for a low cost RT-PCR/PCR cartridge. Embodiments of the present invention comprise a chamber formed within a printed circuit board which is configured to receive an aqueous fluid containing the sample to be analyzed. A heater is also formed within or on the circuit board to heat the aqueous fluid through different temperature steps.

Printed circuit boards are formed by the sequential lamination of metal (copper) coated polymer layers. For rigid boards, materials such as FR4 (epoxy-impregnated fiberglass) are typical. For flexible boards, polyimide-based films are the most common. After each layer is bonded to the board, patterns (traces) are defined with photolithography; the unwanted copper is then etched away. Thus, by sequential bonding and photolithography/etch processes; boards with complex, multilayer patterns may be formed. Connections between layers (vias) are typically formed by drilling holes either through the board, or through selected layers, and then plating the interior of the resulting hole with copper.

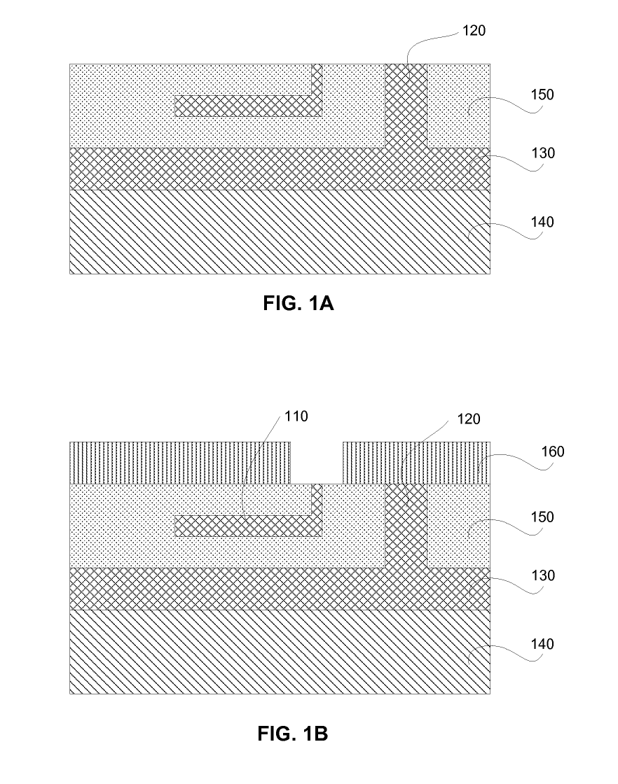

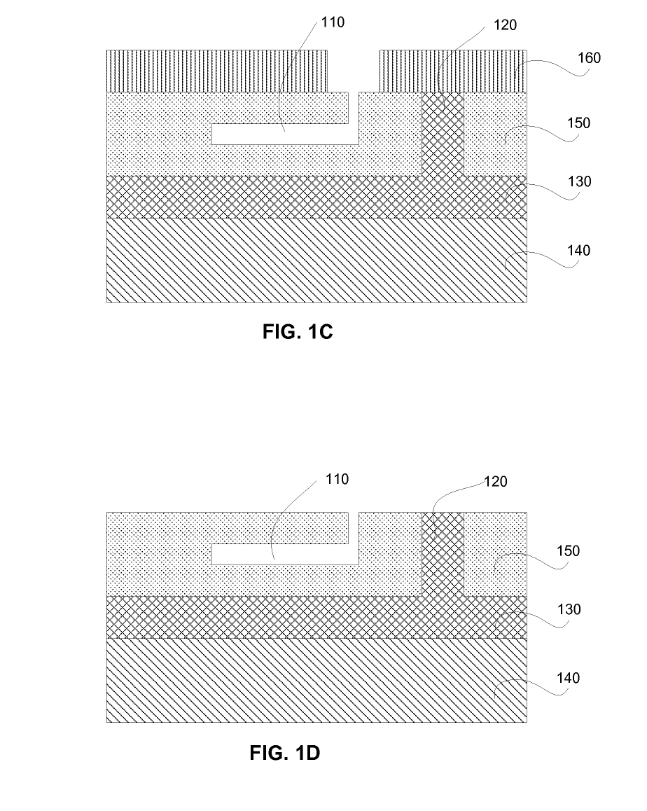

To create a printed circuit board with combined electrical and fluidic functionality, a board is designed with certain elements designated for fluidic use. FIGS. 1A to 1D illustrate the elements formed within a printed circuit board to provide the desired functionality and steps used to form those elements. FIG. 1A shows an initial printed circuit board structure having an FR4 layer 140 upon which an electrical layer 130 is deposited. The electrical layer 130 preferably comprises copper, but may comprises other electrically conductive material. A polyimide layer 150 is deposited on top of the electrical layer 120. A fluidic cavity 110 comprising copper or other sacrificial material is formed within the polyimide layer 150. An electrically conductive vertical structure 120 may also be formed within the polyimide layer to provide electrical contact to the electrical layer 120. The copper or other electrically conductive material in the electrical layer 120 or vertical structure 120 may also function as a sacrificial layer. As described below, an etch process is used on the initial printed circuit board structure to form the desired elements.

To preserve the functionality of the electrical portions of the circuit board, the etch process must be selective. This can be accomplished by physically masking the electrical portions of the board (photoresist, dry film resist, and plastic laminate) before etching. FIG. 1B shows the deposition of an etch mask layer 160 that exposes the copper within the fluidic cavity 110. The copper in the fluidic structures is then etched away leaving a hollow chamber embedded within the printed circuit board. FIG. 1C shows the formation of the hollow fluidic chamber 110 that results from etching. Etching can be performed with a variety of aqueous etchants (ferric chloride, sodium persulphate, etc). The application of an ultrasonic acoustic field may enhance the etch rate, particularly in deeply embedded, thin structures. The mask layer can then be removed with a PCB compatible resist stripper such as sodium hydroxide. FIG. 1D shows the resultant printed circuit board structure after the etch layer 160 has been removed.

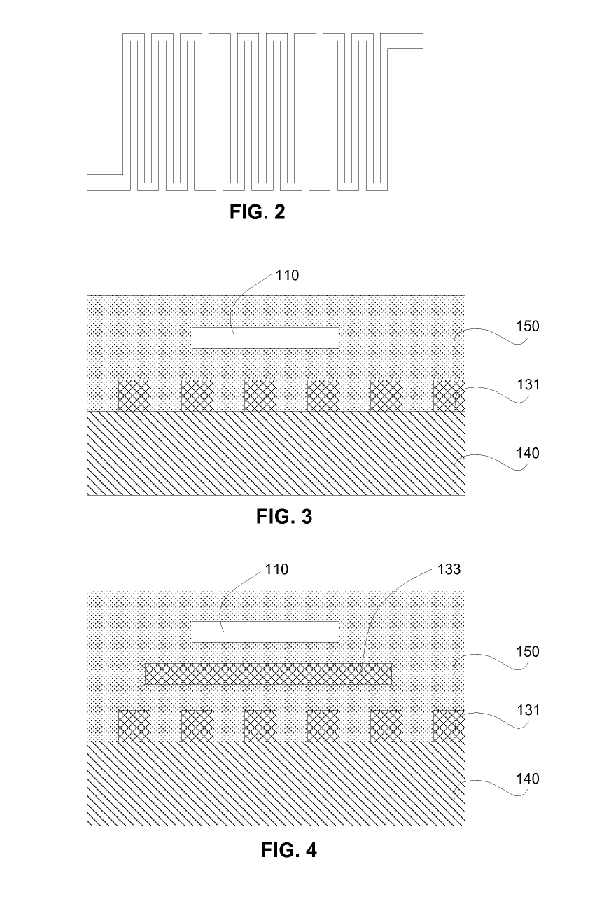

A heater may be implemented in an electrical layer 130 of a PCB by using a long copper trace folded in a serpentine path as a resistor. FIG. 2 illustrates a trace that may be used to implement a heater. Formation of a heater in an electrical layer creates an area on the PCB which can be selectively heated. For typical device sizes, the available resistances are on the order of an Ohm. For example, a half inch by half inch square resistor fabricated on a 1 oz copper (35 microns thick) layer, with a 6 mil wide trace on 6 mil spacing gives a resistance of 1.7 Ohms. When driven at 3 amps, this resistor allows the generation of 8.7 W of thermal power.

FIG. 3 shows a heater structure 131 disposed beneath the fluidic chamber 110 in the polyimide layer 150. When separated from a planar fluidic chamber 131 by a thin layer of polyimide 150, the heater 131 is thermally coupled to the chamber 110. While polyimide has a poor thermal conductivity (0.52 W/m·K), because the separation layer between the chamber and heater is so thin (typically 25 to 100 microns), heat can still be efficiently transmitted from the heater 131 to the chamber 110. The temperature profile within the chamber will be affected by the physical layout of the resistor layer—heat is only generated by the resistor traces. The resulting unevenness may be mitigated by reducing the resistor trace and spacing dimensions.

The temperature profile created by the heater 131 may also be smoothed by the addition of a second copper layer placed between the resistor and chamber. FIG. 4 shows a heat smoothing layer 133 made of copper disposed between the heater 131 and the fluidic chamber 110. Since copper is an efficient conductor of heat (it has a thermal conductivity of 401 W/m·K), this heat smoothing layer 133 will be fairly isothermal, and thus evenly conduct heat from the heater 131 into the chamber 110. Those skilled in the art will understand that materials other than copper may be used for the heater 131 and/or the heat smoothing layer 133.

Temperature within the fluidic chamber may be controlled by controlling the current applied to the embedded resistor. Since the resistivity of copper changes with temperature (about 7 ppm/° K), a resistance measurement of the heater resistor, calibrated for the thermal coupling between the chamber and heater, allows for the indirect electrical measurement and control of the chamber temperature. Alternatively, an external measurement of the fluidic chamber's temperature may be made by optically (via infrared thermometer) or by the attachment of a thermocouple. An analog feedback loop may then be used to stabilize the temperature of the chamber. The temperature may be controlled electronically in conjunction with a temperature sense—current control feedback loop to force the fluidic chamber through a series of temperature cycles.

The external face of the fluidic chamber as fabricated in this process will be polyimide, or another transparent polymer. In the case of polyimide, the optical absorbance is significant at shorter wavelengths (about 150/cm at a wavelength of 500 nm). The chamber wall is so thin, however, that an optical pump can still be efficiently transmitted into the chamber (for a 50 micron thick chamber wall, 47% of the light would be coupled to the fluid). The wavelengths produced by fluorophores are longer than the pump wavelength, and suffer less absorbance. When filled with an appropriate PCR solution with fluorescent tags, optical measurement of the nucleic acid concentration within the chamber may thus be performed. The PCR solution, or master-mix, may be lyophilized inside the fluidic chamber during the fabrication of the PCB discussed above. Alternatively, the PCR solution may be directed into the fluidic chamber after the PCB is fabricated and the PCR solution lyophilized in situ. When a PCR analysis is to be run, a sample analyte suspended in a solution would be directed into the fluidic chamber to reconstitute the PCR solution and the analysis performed. In another embodiment, the PCR solution and the sample analyte may be directed into the fluidic chamber as separate solutions or a combined solution. Fluidic inlets or other means may be used to direct solutions into the fluidic chamber.

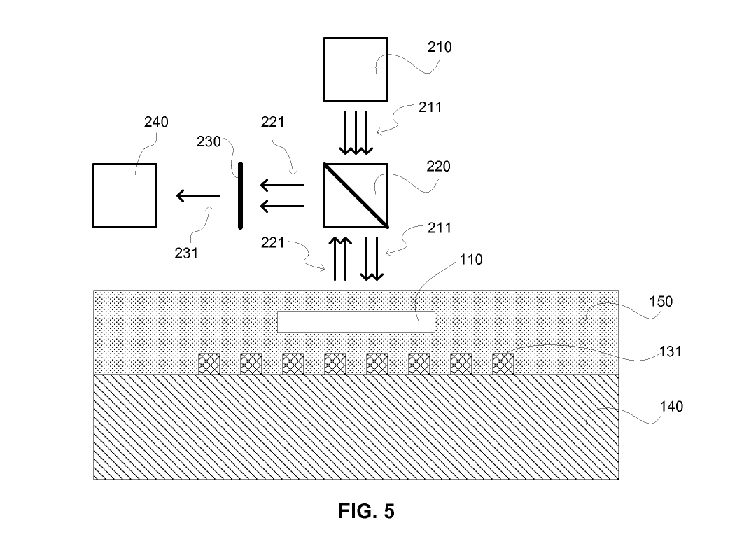

FIG. 5 shows a RT-PCR analysis system 200 utilizing a PCB-based fluidic chamber. The basic elements of the system 200 shown in FIG. 5 are a source 210 (e.g., LED, laser, etc.) to couple the optical pump energy 211 into the chamber 110, a beamsplitter 220, or wavelength selective dichroic mirror to couple a fluorescence signal 221 out of the chamber 110, a filter 230 to remove residual pump or polyimide autofluorescence signal 231 from the optical signal 221, and a detector 240 (e.g., photomultiplier, avalanche photodiode, etc.) to convert the collected light into an electrical measurement.

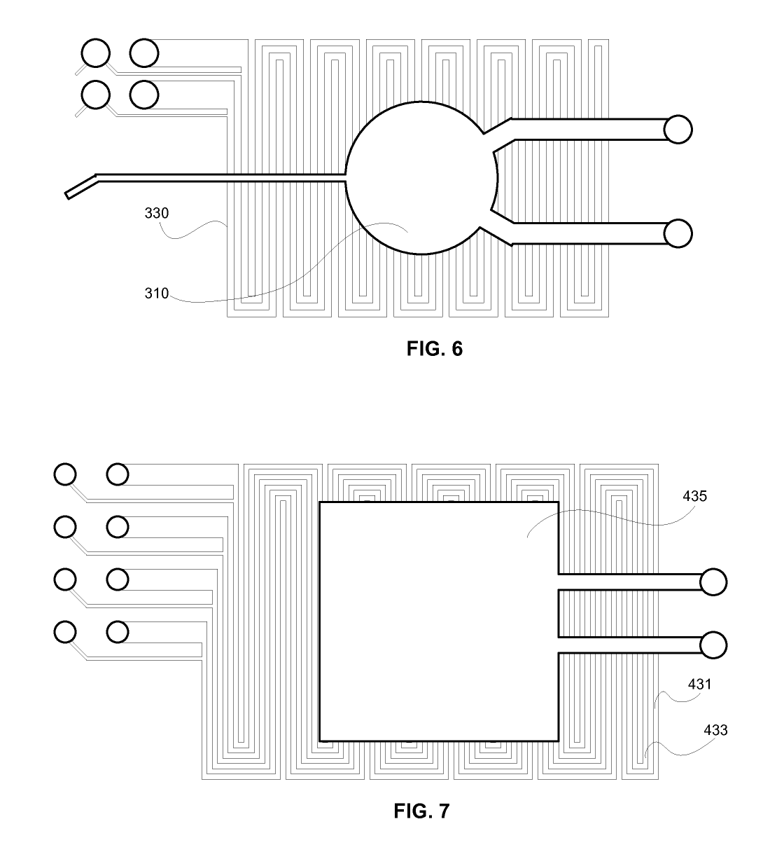

Exemplary devices used for testing were fabricated in a mixed FR4/polyimide/copper printed circuit board material system. The layout of one such device is shown in FIG. 6. FIG. 6 shows the traces 330 used for a heating element and a central chamber 310 fabricated to receive an aqueous solution. Note that the traces 330 shown in FIG. 6 are interlaced in a manner to allow a current supply and return to be applied at the same side of the device. In the device shown in FIG. 6, when the central chamber was filled with distilled water and the heater was connected to a current source, it was possible to generate steam in the fluidic chamber in under 10 seconds. Heating such a chamber filled with an aqueous solution for PCR (to the 95° C. DNA melting temperature) should be accomplished in less time.

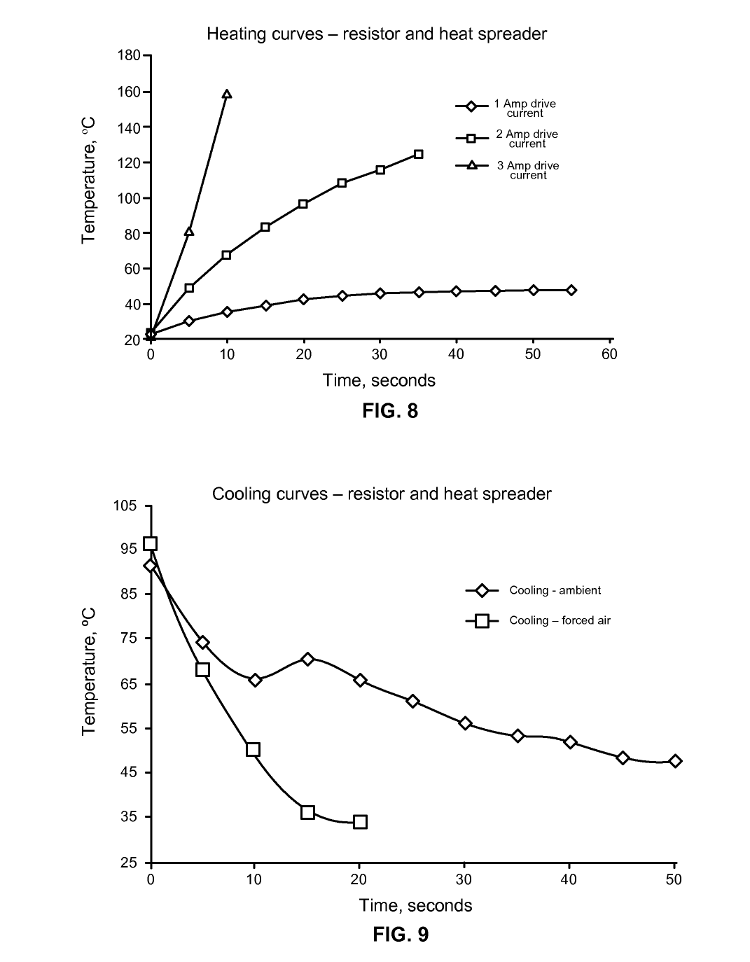

FIG. 7 shows a device in which a hollow central chamber was not created. Instead, the device shown in FIG. 7 was unetched—the large square chamber 435 was tested as a heat spreader. The heater design in this device is comprised of two interlaced resistors 431, 433, which, when wired in parallel, have a total resistance of 0.7 ohms. An infrared thermometer directed at the copper heat spreader structure 435 was used to measure the temperature of the spreader as power was applied to the resistor layers. Temperature vs. time curves were measured for heating and cooling in an ambient temperature of 25 C. FIG. 8 shows the temperature curves for heating and FIG. 9 shows the temperature curves for cooling. For the cooling curves, a set of data was taken with forced air convection to speed the cooling process. Analysis of these curves suggests that heating from the nominal 50° C. to 95° C. points in the PCR cycle may be accomplished in 5 seconds, and cooling in 10 seconds. Even allowing for stabilization time, and the delays associated with a feedback loop, it should still be possible to complete a full PCR temperature cycle in less than 20 seconds.

The microfluidic printed circuit board platform lends itself to cheap/scalable fabrication. Furthermore, the biocompatibility of the PCB material systems such as polyimide allows for the manufacturing of low-cost polymerase chain reactors. Coupled with cheap, non-disposable optics and electronics, this is the foundation of a simple total analysis platform that can be deployed for in-field testing of common diseases such as swine-flu, avian-flu, and HIV. The economics of PCB manufacturing lends itself for cheap fabrication of the PCB materials. The microfluidic PCB may be constructed for multiple uses, requiring appropriate cleaning between uses. However, due to the low cost of the microfluidic PCB, the PCB may be manufactured with an intended single-use disposable protocol. Further, as indicated above, the microfluidic PCB may be constructed as a cartridge containing a lyophilized cocktail (including PCR master-mix with the necessary PCR-product detection molecules). The sample analyte would then be introduced into the fluidic chamber within the cartridge at the time that the PCR analysis is to be performed.

Those skilled in the art understand that the printed circuit board structure described above may be formed using printed circuit board fabrication techniques discussed above, i.e., sequential bonding and photolithography/etch processes, or other techniques known in the art, such as thin-film lamination techniques. Such fabrication techniques may also support the fabrication of the fluidic inlets for the introduction of solutions into the fluidic chamber or other techniques may be used to form the ports or inlets into the fluidic chamber. Also, fabrication techniques may also allow the integration of some of the separate components described above, such as the mirrors or filters, with the microfluidic printed circuit board to provide increased utility or lower cost.

The foregoing Detailed Description of exemplary and preferred embodiments is presented for purposes of illustration and disclosure in accordance with the requirements of the law. It is not intended to be exhaustive nor to limit the invention to the precise form or forms described, but only to enable others skilled in the art to understand how the invention may be suited for a particular use or implementation. The possibility of modifications and variations will be apparent to practitioners skilled in the art.

No limitation is intended by the description of exemplary embodiments which may have included tolerances, feature dimensions, specific operating conditions, engineering specifications, or the like, and which may vary between implementations or with changes to the state of the art, and no limitation should be implied therefrom. In particular it is to be understood that the disclosures are not limited to particular compositions or biological systems, which can, of course, vary. This disclosure has been made with respect to the current state of the art, but also contemplates advancements and that adaptations in the future may take into consideration of those advancements, namely in accordance with the then current state of the art. It is intended that the scope of the invention be defined by the Claims as written and equivalents as applicable. It is also to be understood that the terminology used herein is for the purpose of describing particular embodiments only, and is not intended to be limiting. Reference to a claim element in the singular is not intended to mean “one and only one” unless explicitly so stated. As used in this specification and the appended claims, the singular forms “a,” “an,” and “the” include plural referents unless the content clearly dictates otherwise. The term “several” includes two or more referents unless the content clearly dictates otherwise. Unless defined otherwise, all technical and scientific terms used herein have the same meaning as commonly understood by one of ordinary skill in the art to which the disclosure pertains.

Moreover, no element, component, nor method or process step in this disclosure is intended to be dedicated to the public regardless of whether the element, component, or step is explicitly recited in the Claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “comprising step(s) for . . . .”

A number of embodiments of the disclosure have been described. Nevertheless, it will be understood that various modifications may be made without departing from the spirit and scope of the present disclosure. Accordingly, other embodiments are within the scope of the following claims.

What is claimed is:

1. A printed circuit board structure comprising:
a first layer;
a second layer disposed on the first layer, wherein the second layer comprises one or more electrical interconnections; and
a third layer disposed on the first layer or the second layer or on the first and second layer, wherein the third layer comprises an enclosed planar chamber, wherein the enclosed planer chamber is configured to receive an aqueous solution.

2. The printed circuit board structure according to claim 1, wherein the enclosed planar chamber is formed by depositing metal in the third layer and then removing the metal by an etch removal process.

3. The printed circuit board structure according to claim 1, wherein the third layer comprises optically transparent material.

4. The printed circuit board structure according to claim 3, wherein the third layer comprises polyimide.

5. The printed circuit board structure according to claim 1, wherein the second layer comprises a heating element.

6. The printed circuit board structure according to claim 5, wherein the heating element comprises one or more traces having a serpentine path from an electrical source to an electrical return.

7. The printed circuit board structure according to claim 5, wherein a heat spreading element is disposed between the heating element and the enclosed planer chamber.

8. The printed circuit board structure according to claim 7, wherein the heat spreading element comprises a metal layer.

9. The printed circuit board structure according to claim 1, further comprising a lyophilized polymerase chain reaction solution contained within the enclosed planar structure.

10. A system for real time polymerase chain reaction analysis comprising:
a printed circuit board cartridge comprising:
a first layer comprising a heating element; and
a second layer in thermal communication with the first layer, wherein the second layer comprises an enclosed planar chamber having an optically accessible outer face, wherein the enclosed planer chamber is configured to receive an aqueous solution,
an optical source configured to direct optical energy into the enclosed planer chamber; and,
an optical monitor configured to monitor optical energy radiated from the enclosed planer chamber.

11. The system according to claim 10, further comprising an electrical source coupled to the heating element, wherein the electrical source is controlled to control temperature of the enclosed planer chamber.

12. The system according to claim 11, wherein the electrical source is controlled to cycle temperature of the enclosed planer chamber through selected temperatures.

13. The system according to claim 11, further comprising a heat spreading element disposed between the heating element and the enclosed planer chamber.

14. The system according to claim 10, wherein the heating element comprises one or more traces having a serpentine path from an electrical source to an electrical return.

15. The system according to claim 13, wherein the heat spreading element comprises a metal layer.

16. The system according to claim 10, wherein the printed circuit board structure further comprises a lyophilized polymerase chain reaction solution contained within the enclosed planar structure.

17. A method for forming a temperature controlled fluidic chamber comprising:
depositing an electrical layer on a base layer to form a resistive heating element;
depositing a polyimide layer on the base layer or the electrical layer or the base layer and the electrical layer;
depositing metal within the polyimide layer to form a planar structure; and
removing the metal from the planar structure to form a planar chamber within the polyimide layer.

18. The method according to claim 17, wherein the resistive heating element comprises one or more serpentine metal traces.

19. The method according to claim 17, further comprising depositing a metal layer between the resistive heating element and the planer structure.

20. The method according to claim 17, wherein the method comprises forming a temperature controlled fluidic chamber for polymerase chain reaction analysis and the method further comprises directing a polymerase chain reaction solution into the planar chamber and lyophilizing the polymerase chain reaction solution.
